(19) United States
(12) Patent Application Publication
(10) Pub. No.: US 2025/0241916 A1
(43) Pub. Date: Jul. 31, 2025

(54) TYROSINE KINASE INHIBITORS

(71) Applicants: Nuvectis Pharma, Inc., Fort Lee, NJ (US); UNIVERSITY COURT OF THE UNIVERSITY OF EDINBURGH, Edinburgh (GB)

(72) Inventors: Enrique Poradosu, Fort Lee, NJ (US); Asier Unciti-Broceta, Edinburgh (GB)

(21) Appl. No.: 18/854,263

(22) PCT Filed: Apr. 6, 2023
(86) PCT No.: PCT/US2023/017704

§ 371 Date: Oct. 4, 2024

Related U.S. Application Data
(60) Provisional application No. 63/328,348, filed on Apr. 7, 2022.

(51) Int. Cl. A61K 31/519; A61K 9/00; A61P 35/00

(52) U.S. Cl. CPC A61K 31/519; A61K 9/0053; A61P 35/00

(57) ABSTRACT

The disclosure is in part directed to a method of treating a cancer, e.g., a B-cell cancer, in a patient in need thereof, comprising administering to the patient an effective amount of a disclosed tyrosine kinase inhibitor. In some embodiments, the cancer harbors a BTK mutation.

### CROSS-REFERENCE TO RELATED APPLICATIONS

This application is the National Stage of International (PCT) Patent Application No. PCT/US2023/017704, filed on Apr. 6, 2023, which claims the benefit of, and priority to, U.S. Provisional Application Ser. No. 63/328,348 filed on Apr. 7, 2022, each of which is incorporated herein by reference in its entirety.

### BACKGROUND

Bruton's tyrosine kinase (BTK) inhibition is an emerging strategy for treatment of B-cell malignancies. Ibrutinib is the most studied BTK inhibitor and the first in this new class approved by the US Food and Drug Administration (FDA) and the European Medicines Agency (EMA). Resistance to ibrutinib treatment has been attributed to the selection of cells carrying a pathogenic mutant altering BTK. The most common resistance variation results in a cysteine (C) to serine (S) substitution at position 481, which prevents the covalent binding of ibrutinib to the thiol group located at the ATP-binding site. When this alteration is introduced into the germline of mice, B-cell development remains normal, demonstrating functional interchangeability. The BTK variants C481F, C481G, C481R and C481Y are enriched in some CLL patients, but occur at much lower frequency than C481S.

Acalabrutinib is a second-generation BTK inhibitor, which covalently binds to wildtype C481. It has higher selectivity and reduces the number of adverse effects compared to ibrutinib. Acalabrutinib has been approved by the FDA for the treatment of mantle cell lymphoma (MCL) and CLL/small lymphocytic leukemia. Zanubrutinib is also a more selective, irreversible BTK inhibitor and FDA approved for the treatment of MCL. Zanubrutinib shows potent preclinical activity and minimal off-target effects in patients with Waldenström macroglobulinemia.

Reversible, non-covalent inhibitors are also selective for BTK, and since they do not bind to C481, the inhibition is likely to be at least partially maintained in presence of the C481S variant. Non-covalent inhibitors have shown high capacity against BTK variants including C481R and T474L/M in in vitro assays. The non-covalent BTK inhibitor fenebrutinib was demonstrated to be safe and has been used in phase I studies with equivalent BTK inhibition shown for wild-type and the C481S variant.

Accordingly, there is a need for the development of BTK inhibitors and methods for the treatment of diseases or conditions responsive to BTK inhibition, e.g., for the treatment of B-cell malignancies, reduce side effects and overcome resistance toward e.g., ibrutinib treatment.

### SUMMARY

The present disclosure is directed, at least in part, to methods of treating cancer using compounds that modulate, e.g., inhibit, Bruton's tyrosine kinase (BTK). For example, disclosed herein is a method of treating a cancer in a patient in need thereof, comprising administering to the patient an effective amount of a Bruton's tyrosine kinase (BTK) inhibitor represented by Formula I:

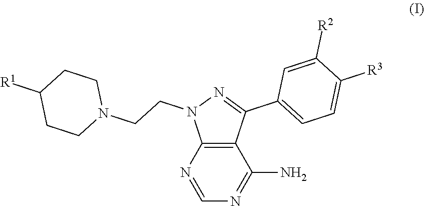

or a pharmaceutically acceptable salt or stereoisomer thereof, wherein:
R¹ is selected from the group consisting of NHR^{a} and NR^{a}R^{b};
R^{a} and R^{b} are each independently selected from the group consisting of C₁-C₆alkyl, C₂-C₆alkenyl, C₃-C₆alkynyl, C₃-C₆cycloalkyl and C₃-C₆cycloalkenyl; or R^{a} and R^{b}, together with the nitrogen to which they are attached, may be joined to form C₃-C₆heterocyclyl;
R² is selected from the group consisting of OR¹¹, hydrogen, halo, NHR¹¹, C₁-C₆alkyl, C₂-C₆alkenyl and C₂-C₆alkynyl;
R³ is selected from the group consisting of NHCO₂R⁴, C₁-C₆alkyl, C₂-C₆alkenyl, C₂-C₆alkynyl, aryl, halo, aryloxy, NH(CO)NR⁵R⁶, NH(CO)R⁷, NH-C₁-C₆alkyl, NH-C₂-C₆alkenyl, NH(CH₂)ₙ-aryl, (CH₂)ₚ-heteroaryl, (CH₂)_{q}CO₂R⁸, (CH₂)ᵣCOR⁹ and NHSO₂R¹⁰; wherein each C₁-C₆alkyl, C₂-C₆alkenyl, aryl or heteroaryl moiety in the aforementioned list is optionally further substituted by one or more groups each independently selected from the group consisting of C₁-C₆alkyl, halo, OH, NR^{c}R^{d}, CONR^{c}R^{d}, C₁-C₆alkoxy, aryloxy, and CO₂H;
R⁴ to R¹¹ are each independently selected from the group consisting of C₁-C₆alkyl, C₂-C₆alkenyl and aryl;
R^{c} and R^{d} are each independently selected from the group consisting of hydrogen, C₁-C₆alkyl and phenyl; and
n, p, q, and r are each independently selected from 0, 1, 2, 3, 4, 5 and 6.

In some embodiments, the cancer is a B-cell cancer, for example, a cancer selected from the group consisting of chronic lymphocytic leukemia, small lymphocytic leukemia, mantle cell lymphoma, non-Hodgkin's lymphoma, marginal zone lymphoma, and Waldenström macroglobulinemia.

Further disclosed herein is a method of treating a cancer alleviated by the selective inhibition of BTK in a patient in need thereof, comprising administering to the patient an effective amount of a tyrosine kinase inhibitor. In some embodiments, the cancer harbors a BTK mutation, for example, the cancer has been identified as having a BTK mutation, for example, a C481 mutation.

In some embodiments, the tyrosine kinase inhibitor is an inhibitor of BTK. In some embodiments, a BTK inhibitor for use in the methods described herein may be for example, tert-butyl (4-(4-amino-1-(2-(4-(dimethylamino)piperidin-1-yl) ethyl)-1H-pyrazolo[3,4-d]pyrimidin-3-yl)-2-methoxyphenyl)carbamate, or a pharmaceutically acceptable salt thereof, and is represented by:

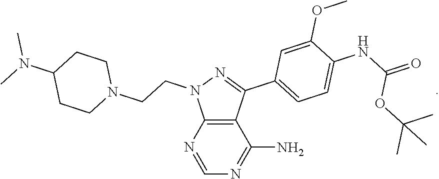 [SMILES: COc1cc(-c2nn(CCN3CCC(N(C)C)CC3)c3ncnc(N)c23)ccc1NC(=O)OC(C)(C)C]

For example, disclosed herein is a method of treating a cancer harboring a BTK mutation, e.g., a cancer identified as having a BTK mutation, in a patient in need thereof, comprising administering to the patient an effective amount of a tyrosine kinase inhibitor, e.g., tert-butyl (4-(4-amino-1-(2-(4-(dimethylamino)piperidin-1-yl)ethyl)-1H-pyrazolo[3,4-d]pyrimidin-3-yl)-2-methoxyphenyl)carbamate, or a pharmaceutically acceptable salt thereof.

Further disclosed herein is a method of treating a B-cell cancer harboring a BTK C481 mutation in a patient in need thereof, comprising administering to the patient an effective amount of tert-butyl (4-(4-amino-1-(2-(4-(dimethylamino)piperidin-1-yl)ethyl)-1H-pyrazolo[3,4-d]pyrimidin-3-yl)-2-methoxyphenyl)carbamate, or a pharmaceutically acceptable salt thereof, wherein the cancer is resistant to treatment with ibrutinib, acalabrutinib, zanubrutinib, and/or fenebrutinib.

Also provided herein is a method of treating squamous cell carcinoma in a patient identified as having said carcinoma, and in need of treatment, comprising administering to the patient an effective amount of a tyrosine kinase inhibitor represented by

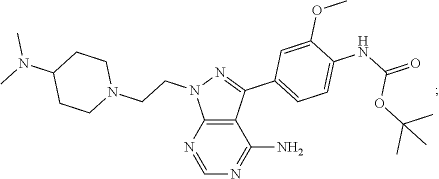 [SMILES: COc1cc(-c2nn(CCN3CCC(N(C)C)CC3)c3ncnc(N)c23)ccc1NC(=O)OC(C)(C)C]

or a pharmaceutically acceptable salt thereof;
wherein the squamous cell carcinoma is selected from the group consisting of cutaneous squamous cell carcinoma, esophageal squamous cell carcinoma, tongue squamous cell carcinoma, head and neck squamous cell carcinoma, squamous cell carcinoma of the vulva, and lung squamous cell carcinoma.

### BRIEF DESCRIPTION OF THE DRAWINGS

FIG. 1 depicts a box and dot plot demonstrating significant sensitivity of pan cancer squamous lines to Compound A (p<0.05).

FIG. 2 shows a statistical association of genetic transformations in 38 cancer genes with shifts in sensitivity to Compound a (as measured by ¹⁰logIC₅₀). A shift to the left represents increased sensitivity. Size of circles represents number of cell lines with alteration (minimum of cell lines per alteration).

FIG. 3 shows a ¹⁰logIC₅₀ distribution of Compound A and FAT1 mutation status. (0=Wild type, 1=Mutant, p=0.16).

FIG. 4 shows tumour volumes over time in the KYSE70 (esophageal squamous cell carcinoma) xenograft model, by treatment group (vehicle or Compound A).

FIG. 5 shows tumour volumes over time in the Cal27 (tongue squamous cell carcinoma) xenograft model, by treatment group (vehicle or Compound A).

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “alkyl,” as used herein, refers to a saturated straight-chain or branched hydrocarbon, such as a straight-chain or branched group of 1-6, 1-5, 1-4, or 1-3 carbon atoms, referred to herein as C₁-C₆ alkyl, C₂-C₆ alkyl, C₁-C₄ alkyl, C₁-C₃ alkyl, respectively. For example, “C₁-C₆ alkyl” refers to a straight-chain or branched saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl. In another example, “C₂-C₆ alkyl” refers to a straight-chain or branched saturated hydrocarbon containing 1-5 carbon atoms. Examples of a C₂-C₆ alkyl group include, but are not limited to, ethyl, propyl, butyl, pentyl, hexyl, isopropyl, isobutyl, sec-butyl, tert-butyl, isopentyl, and neopentyl. In a further example, “C₁-C₄ alkyl” refers to a straight-chain or branched saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂-C₅alkenyl, C₂-C₆alkenyl, and C₃-C₄alkenyl, respectively. Exemplary alkenyl groups include, but are not limited to, vinyl, allyl, butenyl, pentenyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁-C₅alkoxy, C₁-C₆alkoxy, and C₂-C₆alkoxy, respectively. Exemplary alkoxy groups include, but are not limited to methoxy, ethoxy, isopropoxy, etc.

The term “cycloalkyl,” as used herein, refers to a monocyclic saturated or partially unsaturated hydrocarbon ring (carbocyclic) system, for example, where each ring is either completely saturated or contains one or more units of unsaturation, but where no ring is aromatic. A cycloalkyl can have 3-6 or 4-6 carbon atoms in its ring system, referred to herein as C₃-C₆ cycloalkyl or C₄-C₆ cycloalkyl, respectively. Exemplary cycloalkyl groups include, but are not limited to, cyclohexyl, cyclohexenyl, cyclopentyl, cyclopentenyl, cyclobutyl, and cyclopropyl.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

The terms “halo” and “halogen,” as used herein, refer to fluoro (F), chloro (Cl), bromo (Br), and/or iodo (I).

The terms “hydroxy” and “hydroxyl” as used herein refers to the radical —OH.

The term “hetero” when used to describe a compound or a group present on a compound means that one or more carbon atoms in the compound or group have been replaced by one or more heteroatoms. The term “heteroatom” as used herein means an atom of any element other than carbon or hydrogen. Examples of heteroatoms include, but are not limited to nitrogen, oxygen and sulfur.

Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g, cycloheteroalkenyl, and the like having from 1 to 5, and particularly from 1 to 3 heteroatoms.

The terms “heteroaryl” or “heteroaromatic group” as used herein refers to an aromatic 5-10 membered ring system containing one or more heteroatoms, for example one to three heteroatoms, such as nitrogen, oxygen, and sulfur. The term may also be used to refer to a 5-7 membered monocyclic heteroaryl or an 8-10 membered bicyclic heteroaryl. Where possible, said heteroaryl ring may be linked to the adjacent radical though carbon or nitrogen. Examples of heteroaryl rings include but are not limited to furan, thiophene, pyrrole, pyrrolopyridine, indole, thiazole, oxazole, isothiazole, isoxazole, imidazole, benzoimidazole, imidazopyridine, pyrazole, triazole, pyridine or pyrimidine, etc.

The terms “heterocyclyl,” “heterocycle,” or “heterocyclic group” are art-recognized and refer to saturated or partially unsaturated 4-10 membered ring structures, whose ring structures include one to three heteroatoms, such as nitrogen, oxygen, and sulfur. Where possible, heterocyclyl rings may be linked to the adjacent radical through carbon or nitrogen. The term may also be used to refer to 4-10 membered saturated or partially unsaturated ring structures that are bridged, fused or spirocyclic ring structures, whose ring structures include one to three heteroatoms, such as nitrogen, oxygen, and sulfur. Examples of heterocyclyl groups include, but are not limited to, pyrrolidine, piperidine, morpholine, thiomorpholine, piperazine, oxetane, azetidine, tetrahydrofuran, dihydrofuran, dihydropyran, tetrahydropyran, etc. In some embodiments, the heterocycle is a spiro heterocycle. In some embodiments, the heterocycle is a bridged heterocycle. “Spiro heterocyclyl,” or “spiro heterocycle” refers to a polycyclic heterocyclyl with rings connected through one common atom (called a spiro atom), wherein the rings have one or more heteroatoms selected from the group consisting of N, O, and S(O)ₘ (wherein m is an integer of 0 to 2) as ring atoms.

“SRC family,” “SRC kinase family” and “SRC family of kinases” refer to a family of non-receptor tyrosine kinases whose members include, but not limited to, for example, BLK, FGR, FRK, FYN, HCK, LCK, LYN, SRC and YES.

“Treating” includes any effect, e.g., lessening, reducing, modulating, or eliminating, that results in the improvement of the condition, disease, disorder and the like. For example, “treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms. It is to be appreciated that references to “treating” or “treatment” include prophylaxis as well as the alleviation of established symptoms of a condition.

The term “disorder” refers to and is used interchangeably with, the terms “disease,” “condition,” or “illness,” unless otherwise indicated.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the present disclosure is desirably a mammal in which treatment, for example, of a cancer or a blood disorder is desired. “Modulation” includes antagonism (e.g., inhibition), agonism, partial antagonism and/or partial agonism.

In the present specification, the terms “effective amount” or “therapeutically effective amount” means the amount of the subject compound that will elicit the biological or medical response of a tissue, system or animal, (e.g., mammal or human) that is being sought by the researcher, veterinarian, medical doctor or other clinician. The compounds of the present disclosure are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, e.g., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure can contain one or more chiral centers and/or double bonds and therefore, can exist as stereoisomers, such as geometric isomers, and enantiomers or diastereomers. The term “stereoisomers,” when used herein, consists of all geometric isomers, enantiomers and/or diastereomers of the compound. For example, when a compound is shown with specific chiral center(s), the compound depicted without such chirality at that and other chiral centers of the compound are within the scope of the present disclosure, i.e., the compound depicted in two-dimensions with “flat” or “straight” bonds rather than in three dimensions, for example, with solid or dashed wedge bonds. Stereospecific compounds may be designated by the symbols “R” or “S,” depending on the configuration of substituents around the stereogenic carbon atom. The present disclosure encompasses all the various stereoisomers of these compounds and mixtures thereof. Mixtures of enantiomers or diastereomers can be designated “(±)” in nomenclature, but a skilled artisan will recognize that a structure can denote a chiral center implicitly. It is understood that graphical depictions of chemical structures, e.g., generic chemical structures, encompass all stereoisomeric forms of the specified compounds, unless indicated otherwise.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures also can be resolved into their component enantiomers by well-known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. See, for example, Carreira and Kvaerno, Classics in Stereoselective Synthesis, Wiley-VCH: Weinheim, 2009.

Geometric isomers, resulting from the arrangement of substituents around a carbon-carbon double bond or arrangement of substituents around a cycloalkyl or heterocycloalkyl, can also exist in the compounds of the present disclosure. The symbol [symbol] denotes a bond that may be a single, double or triple bond as described herein. Substituents around a carbon-carbon double bond are designated as being in the “Z” or “E” configuration, where the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “and/or” is used in this disclosure to mean either “and” or “or” unless indicated otherwise.

As used herein, the words “a” and “an” are meant to include one or more unless otherwise specified. For example, the term “an agent” encompasses both a single agent and a combination of two or more agents.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

### Methods

In some embodiments, disclosed herein is a method of treating a cancer in a patient in need thereof, comprising administering to the patient an effective amount of a Bruton's tyrosine kinase (BTK) inhibitor represented by Formula I:

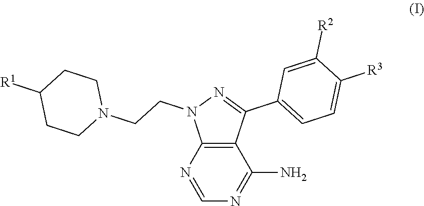

or a pharmaceutically acceptable salt or stereoisomer thereof, wherein:
R¹ is selected from the group consisting of NHR^{a} and NR^{a}R^{b};
R^{a} and R^{b} are each independently selected from the group consisting of C₁-C₆alkyl, C₂-C₆alkenyl, C₃-C₆alkynyl, C₃-C₆cycloalkyl and C₃-C₆cycloalkenyl; or R^{a} and R^{b}, together with the nitrogen to which they are attached, may be joined to form C₃-C₆heterocyclyl;
R² is selected from the group consisting of OR¹¹, hydrogen, halo, NHR¹¹, C₁-C₆alkyl, C₂-C₆alkenyl and C₂-C₆alkynyl;
R³ is selected from the group consisting of NHCO₂R⁴, C₁-C₆alkyl, C₂-C₆alkenyl, C₂-C₆alkynyl, aryl, halo, aryloxy, NH(CO)NR⁵R⁶, NH(CO)R⁷, NH-C₁-C₆alkyl, NH-C₂-C₆alkenyl, NH(CH₂)ₙ-aryl, (CH₂)ₚ-heteroaryl, (CH₂)_{q}CO₂R⁸, (CH₂)ᵣCOR⁹ and NHSO₂R¹⁰; wherein each C₁-C₆alkyl, C₂-C₆alkenyl, aryl or heteroaryl moiety in the aforementioned list is optionally further substituted by one or more groups each independently selected from the group consisting of C₁-C₆alkyl, halo, OH, NR^{c}R^{d}, CONR^{c}R^{d}, C₁-C₆alkoxy, aryloxy, and CO₂H;
R⁴ to R¹¹ are each independently selected from the group consisting of C₁-C₆alkyl, C₂-C₆alkenyl and aryl;
R^{c} and R^{d} are each independently selected from the group consisting of hydrogen, C₁-C₆alkyl and phenyl; and
n, p, q, and r are each independently selected from 0, 1, 2, 3, 4, 5 and 6.

In some embodiments, R^{a} and R^{b} are each independently C₁-C₆alkyl or C₂-C₆alkenyl. For example, in certain embodiments R^{a} and R^{b} are each CH₃. In other embodiments, R^{a} is C₁-C₆alkyl or C₂-C₆alkenyl and R^{b} is hydrogen. For example, in certain embodiments R^{a} is CH₃ and R^{b} is hydrogen.

In further embodiments, R² is C₁-C₆alkoxy or hydrogen. For example, in certain embodiments R² is OCH₃. In other embodiments, R⁴ to R¹¹ are each independently C₁-C₆alkyl.

In some embodiments, R³ is selected from the group consisting of NHCO₂-C₁-C₆alkyl, NHCO-C₁-C₆alkyl, NH(CH₂)ₙ-aryl, NHCONH-C₁-C₆alkyl, (CH₂)ₚ-heteroaryl and (CH₂)_{q}CO₂-C₁-C₆alkyl. For example, in certain embodiments, R³ is selected from the group consisting of NHCO₂-^{t}Bu, NHCOCH₂C(CH₃)₃, NHCH₂phenyl, NHCONH-^{t}Bu, CH₂-(4-methyl-oxazol-2-yl) and CH₂CO₂-^{t}Bu. In certain embodiments, R³ is NHCO₂-^{t}Bu.

In some embodiments, a BTK inhibitor for use in the methods described herein may be for example, tert-butyl (4-(4-amino-1-(2-(4-(dimethylamino)piperidin-1-yl)ethyl)-1H-pyrazolo[3,4-d]pyrimidin-3-yl)-2-methoxyphenyl)carbamate, or a pharmaceutically acceptable salt thereof, and is represented by:

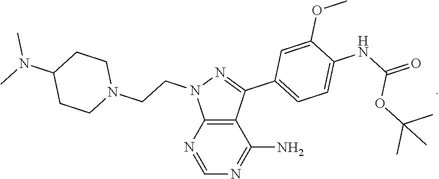 [SMILES: COc1cc(-c2nn(CCN3CCC(N(C)C)CC3)c3ncnc(N)c23)ccc1NC(=O)OC(C)(C)C]

For example, in certain embodiments a BTK inhibitor for use in the methods described herein is

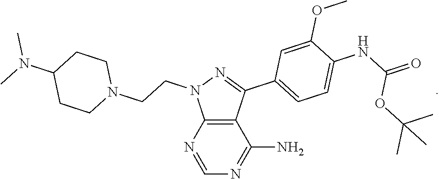 [SMILES: COc1cc(-c2nn(CCN3CCC(N(C)C)CC3)c3ncnc(N)c23)ccc1NC(=O)OC(C)(C)C]

In other embodiments, a BTK inhibitor for use in the methods described herein may be for example, tert-butyl (4-(4-amino-1-(2-(4-(methylamino)piperidin-1-yl)ethyl)-1H-pyrazolo[3,4-d]pyrimidin-3-yl)-2-methoxyphenyl)carbamate, or a pharmaceutically acceptable salt thereof, and is represented by:

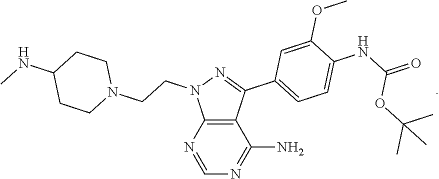 [SMILES: CNC1CCN(CCn2nc(-c3ccc(NC(=O)OC(C)(C)C)c(OC)c3)c3c(N)ncnc32)CC1]

For example, in certain embodiments a BTK inhibitor for use in the methods described herein is

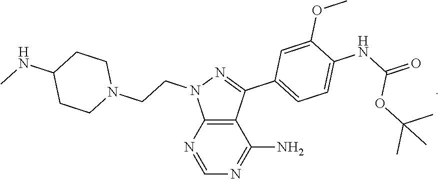 [SMILES: CNC1CCN(CCn2nc(-c3ccc(NC(=O)OC(C)(C)C)c(OC)c3)c3c(N)ncnc32)CC1]

In some embodiments, the cancer is a B-cell cancer. For example, in some embodiments the cancer is selected from the group consisting of chronic lymphocytic leukemia, small lymphocytic leukemia, mantle cell lymphoma, non-Hodgkin's lymphoma, marginal zone lymphoma, and Waldenström macroglobulinemia.

In other embodiments, the cancer harbors a BTK mutation. For example, in some embodiments the cancer has been identified as having a BTK mutation. In certain embodiments, the BTK mutation is a C481 mutation. For example, in certain embodiments the BTK mutation is selected from the group consisting of but not limited to, e.g., a C481F mutation, a C481G mutation, a C481R mutation, a C481S mutation, and a C481Y mutation. In some embodiments, the BTK mutation is a C481S mutation.

In some embodiments, the BTK mutation in the cancer is a result of previously treating the cancer with one or more other cancer therapeutic agents. In certain embodiments, treating the cancer with the one of more other cancer therapeutic agents is no longer effective in treating the cancer. In other embodiments, the one or more other cancer therapeutic agents is selected from the group consisting of but not limited to, for example, ibrutinib, acalabrutinib, zanubrutinib, fenebrutinib, tirabrutinib, tolebrutinib, evobrutinib, pirtobrutinib, spebrutinib.

In some embodiments, the BTK mutation is a heterozygous BTK mutation. In other embodiments, the BTK mutation is a homozygous BTK mutation. In further embodiments, the BTK inhibitor is administered orally. In other embodiments, the BTK inhibitor is administered subcutaneously. In further embodiments, the compound is administered intraperitoneally. In still other embodiments, the BTK inhibitor is administered intravenously.

In some embodiments, the method further and optionally comprises administering one or more additional cancer chemotherapeutic agents. For example, in other embodiments, the method further and optionally comprises administering an additional cancer chemotherapeutic agent.

In certain embodiments, the tyrosine kinase inhibitor is an inhibitor of BTK. In other embodiments, the tyrosine kinase inhibitor further inhibits members of the SRC kinase family, including but not limited to, for example, BLK, FGR, FRK, FYN, HCK, LCK, LYN, SRC and YES.

Also disclosed herein is a method of treating a cancer alleviated by the selective inhibition of BTK in a patient in need thereof, comprising administering to the patient an effective amount of a tyrosine kinase inhibitor represented by:

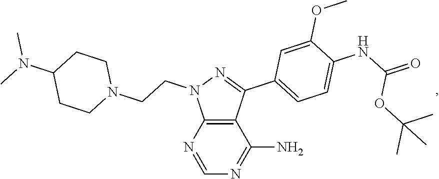 [SMILES: COc1cc(-c2nn(CCN3CCC(N(C)C)CC3)c3ncnc(N)c23)ccc1NC(=O)OC(C)(C)C]

or a pharmaceutically acceptable salt thereof.

In some embodiments, the cancer is selected from the group consisting of chronic lymphocytic leukemia, small lymphocytic leukemia, mantle cell lymphoma, non-Hodgkin's lymphoma, marginal zone lymphoma, and Waldenström macroglobulinemia. In certain embodiments, the cancer harbors a BTK mutation. For example, in some embodiments the BTK mutation is a C481 mutation, e.g., a BTK mutation selected from the group consisting of a C481F mutation, a C481G mutation, a C481R mutation, a C481S mutation, and a C481Y mutation.

Further disclosed herein is a method of treating a cancer harboring a BTK mutation in a patient in need thereof, comprising administering to the patient an effective amount of a tyrosine kinase inhibitor represented by:

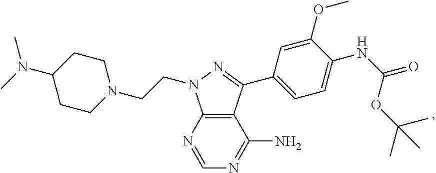 [SMILES: COc1cc(-c2nn(CCN3CCC(N(C)C)CC3)c3ncnc(N)c23)ccc1NC(=O)OC(C)(C)C ; ","]

or a pharmaceutically acceptable salt thereof.

In some embodiments, the cancer is selected from the group consisting of chronic lymphocytic leukemia, small lymphocytic leukemia, mantle cell lymphoma, non-Hodgkin's lymphoma, marginal zone lymphoma, and Waldenström macroglobulinemia. In certain embodiments, the cancer harbors a BTK mutation. For example, in some embodiments the BTK mutation is a C481 mutation, e.g., a BTK mutation selected from the group consisting of a C481F mutation, a C481G mutation, a C481R mutation, a C481S mutation, and a C481Y mutation.

In some embodiments, the BTK mutation in the cancer is a result of previously treating the cancer with one or more other cancer therapeutic agents. In certain embodiments, treating the cancer with the one of more other cancer therapeutic agents is no longer effective in treating the cancer. In other embodiments, the one or more other cancer therapeutic agents is selected from the group consisting of but not limited to, for example, ibrutinib, acalabrutinib, zanubrutinib, fenebrutinib, tirabrutinib, tolebrutinib, evobrutinib, pirtobrutinib, spebrutinib.

Further disclosed herein is a method of treating a B-cell cancer harboring a BTK C481 mutation in a patient in need thereof, comprising administering to the patient an effective amount of tert-butyl (4-(4-amino-1-(2-(4-(dimethylamino)piperidin-1-yl)ethyl)-1H-pyrazolo[3,4-d]pyrimidin-3-yl)-2-methoxyphenyl)carbamate, or a pharmaceutically acceptable salt thereof, wherein the cancer is resistant to treatment with ibrutinib, acalabrutinib, zanubrutinib, fenebrutinib, tirabrutinib, tolebrutinib, evobrutinib, pirtobrutinib, and/or spebrutinib.

In some embodiments, a cancer type disclosed herein, for example, a B-cell cancer, has been identified as having a BTK mutation. Methodologies to detect the BTK mutations contemplated herein are known in the art.

In particular, in certain embodiments, the disclosure provides a method of treating the above medical indications comprising administering to a patient in need thereof an effective amount of a BTK inhibitor disclosed herein. In certain other embodiments, the disclosure provides a method of treating the above medical conditions in a patient in need thereof, comprising orally, subcutaneously, or intravenously administering to the patient a composition comprising a disclosed BTK inhibitor.

Without wishing to be bound to any particular theory, it is believed that compounds of the present disclosure (e.g., tert-butyl (4-(4-amino-1-(2-(4-(dimethylamino) piperidin-1-yl)ethyl)-1 H-pyrazolo[3,4-d]pyrimidin-3-yl)-2-methoxyphenyl) carbamate) are also inhibitors of SRC family kinases, and that the combined effects on both SRC family kinases and BTK (both wildtype and mutant) are likely to be advantageous in treating B cell cancers.

In some embodiments, a method of treatment disclosed herein may produce an antiproliferative effect. In some embodiments, a method of treatment disclosed herein may be a method of treating a proliferative disease or a proliferative disorder. The terms “proliferative disease” and “proliferative disorder” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, cancers, lymphomas, leukemias and solid tumors.

Further disclosed herein is a method of treating squamous cell carcinoma in a patient identified as having said carcinoma, and in need of treatment, comprising administering to the patient an effective amount of a tyrosine kinase inhibitor represented by Formula I:

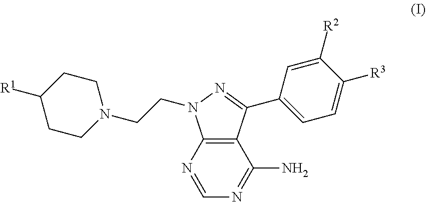

or a pharmaceutically acceptable salt or stereoisomer thereof, wherein:
R¹ is selected from the group consisting of NHR^{a} and NR^{a}R^{b};
R^{a} and R^{b} are each independently selected from the group consisting of C₁-C₆alkyl, C₂-C₆alkenyl, C₃-C₆alkynyl, C₃-C₆cycloalkyl and C₃-C₆cycloalkenyl; or R^{a} and R^{b}, together with the nitrogen to which they are attached, may be joined to form C₃-C₆heterocyclyl;
R² is selected from the group consisting of OR¹¹, hydrogen, halo, NHR¹¹, C₁-C₆alkyl, C₂-C₆alkenyl and C₂-C₆alkynyl;
R³ is selected from the group consisting of NHCO₂R⁴, C₁-C₆alkyl, C₂-C₆alkenyl, C₂-C₆alkynyl, aryl, halo, aryloxy, NH(CO)NR⁵R⁶, NH(CO)R⁷, NH-C₁-C₆alkyl, NH-C₂-C₆alkenyl, NH(CH₂)ₙ-aryl, (CH₂)ₚ-heteroaryl, (CH₂)_{q}CO₂R⁸, (CH₂)ᵣCOR⁹ and NHSO₂R¹⁰; wherein each C₁-C₆alkyl, C₂-C₆alkenyl, aryl or heteroaryl moiety in the aforementioned list is optionally further substituted by one or more groups each independently selected from the group consisting of C₁-C₆alkyl, halo, OH, NR^{c}R^{d}, CONR^{c}R^{d}, C₁-C₆alkoxy, aryloxy, and CO₂H;
R⁴ to R¹¹ are each independently selected from the group consisting of C₁-C₆alkyl, C₂-C₆alkenyl and aryl;
R^{c} and R^{d} are each independently selected from the group consisting of hydrogen, C₁-C₆alkyl and phenyl; and
n, p, q, and r are each independently selected from 0, 1, 2, 3, 4, 5 and 6.

In some embodiments, R^{a} and R^{b} are each independently C₁-C₆alkyl or C₂-C₆alkenyl. For example, in certain embodiments R^{a} and R^{b} are each CH₃. In other embodiments, R^{a} is C₁-C₆alkyl or C₂-C₆alkenyl and R^{b} is hydrogen. For example, in certain embodiments R^{a} is CH₃ and R^{b} is hydrogen.

In further embodiments, R² is C₁-C₆alkoxy or hydrogen. For example, in certain embodiments R² is OCH₃. In other embodiments, R⁴ to R¹¹ are each independently C₁-C₆alkyl.

In some embodiments, R³ is selected from the group consisting of NHCO₂-C₁-C₆alkyl, NHCO-C₁-C₆alkyl, NH(CH₂)ₙ-aryl, NHCONH-C₁-C₆alkyl, (CH₂)ₚ-heteroaryl and (CH₂)_{q}CO₂-C₁-C₆alkyl. For example, in certain embodiments, R³ is selected from the group consisting of NHCO₂-^{t}Bu, NHCOCH₂C(CH₃)₃, NHCH₂phenyl, NHCONH-^{t}Bu, CH₂-(4-methyl-oxazol-2-yl) and CH₂CO₂-^{t}Bu. In certain embodiments, R³ is NHCO₂-^{t}Bu.

For example, is some embodiments the tyrosine kinase inhibitor is selected from the group consisting of:

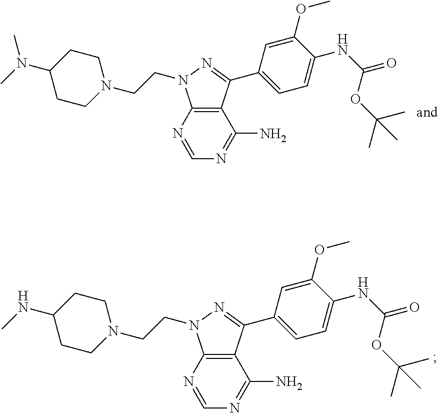

or a pharmaceutically acceptable salt thereof.

In certain embodiments, the tyrosine kinase inhibitor is

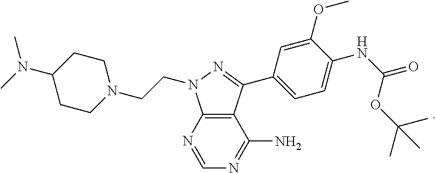 [SMILES: COc1cc(-c2nn(CCN3CCC(N(C)C)CC3)c3ncnc(N)c23)ccc1NC(=O)OC(C)(C)C ; "."]

In some embodiments, the squamous cell carcinoma is selected from the group consisting of, for example, cutaneous squamous cell carcinoma, esophageal squamous cell carcinoma, tongue squamous cell carcinoma, head and neck squamous cell carcinoma, squamous cell carcinoma of the vulva, and lung squamous cell carcinoma.

For example, provided herein is a method of treating squamous cell carcinoma in a patient identified as having said carcinoma, and in need of treatment, comprising administering to the patient an effective amount of a tyrosine kinase inhibitor represented by

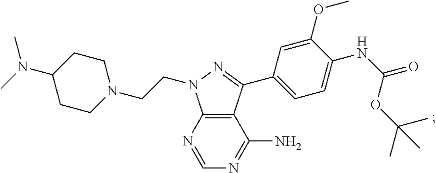 [SMILES: COc1cc(-c2nn(CCN3CCC(N(C)C)CC3)c3ncnc(N)c23)ccc1NC(=O)OC(C)(C)C ; ";"]

or a pharmaceutically acceptable salt thereof;
wherein the squamous cell carcinoma is selected from the group consisting of cutaneous squamous cell carcinoma, esophageal squamous cell carcinoma, tongue squamous cell carcinoma, head and neck squamous cell carcinoma, squamous cell carcinoma of the vulva, and lung squamous cell carcinoma.

The treatments defined herein may be applied as a sole therapy or may involve, in addition to the compound of the invention, conventional surgery, radiotherapy, gene therapy or therapy with a chemotherapeutic agent or a molecularly targeted agent. Such conjoint treatment may be achieved by way of the simultaneous, sequential or separate dosing of the individual components of the treatment. The term “combination” as used herein refers to simultaneous, separate or sequential administration. In some embodiments, “combination” refers to simultaneous administration. In other embodiments, “combination” refers to separate administration. In further embodiments, “combination” refers to sequential administration. Where the administration is sequential or separate, the delay in administering the second component should not be such as to lose the beneficial effect of the combination.

### EXAMPLES

The following non-limiting examples illustrate the disclosure.

tert-Butyl (4-(4-amino-1-(2-(4-(dimethylamino)piperidin-1-yl)ethyl)-1H-pyrazolo[3,4-d]pyrimidin-3-yl)-2-methoxyphenyl)carbamate (Compound A) can be prepared according to the synthetic procedures described in WO 2016/185160, the content of which is incorporated by reference herein. The des-methyl derivative tert-butyl (4-(4-amino-1-(2-(4-(methylamino)piperidin-1-yl)ethyl)-1H-pyrazolo[3,4-d]pyrimidin-3-yl)-2-methoxyphenyl) carbamate (Compound B) can be prepared through a similar synthetic procedure.

### Example 1

### Tyrosine Kinase Inhibition Studies

Compound A was dissolved in dimethylsulfoxide (DMSO), and further diluted with assay buffer to a final concentration of 0.5 μmol/L. Reference compounds for assay control were prepared similarly. For BTK inhibition studies, full-length human BTK [2-659 (end) amino acids of accession number NP_000052] was expressed as N-terminal GST-fusion protein (103 kDa) using baculovirus expressionsystem. GST-BTK was purified by using glutathione sepharose chromatography. For BTK[C481S] inhibition studies, full-length human BTK [2-659 (end) amino acids and C481S of accession number NP_000052] was expressed as N-terminal GST-fusion protein (103 kDa) using baculovirusexpression system. GST-BTK[C481S] was purified by using glutathione sepharosechromatography.

A 4× Substrate/ATP/Metal solution was prepared with buffer (20 mmol/L HEPES, 0.01% Triton X-100, 5 mmol/L DTT, pH 7.5), and a 2× kinase solution was also prepared with buffer (20 mmol/L HEPES, 0.01% Triton X-100, 1 mmol/L DTT, pH 7.5). 5 μL of 4× compound solution, 5 μL of 4× Substrate/ATP/Metal solution, and 10 μL of 2× kinase solution were mixed and incubated in a well of polypropylene 384 well microplate for 1 hour at room temperature. 70 μL of Termination Buffer (QuickScout Screening Assist MSA) was added to the well. The reaction mixture was applied to LabChip™ system, and the product and substrate peptide peaks were separated and quantitated. The kinase reaction was evaluated by the product ratio calculated from peak heights of product (P) and substrate (S) peptides (P/(P+S)).

TABLE 1
Off-chip Mobility Shift Assay (MSA) Conditions

| Kinase | Substrate | ATP | Mg²⁺ | Positive Control |
|---|---|---|---|---|
| BTK | Srctide | 5 μmol/mL | 5 mmol/L | Staurosporine |
| | (1 μmol/mL) | | | |
| BTK[C481S] | Srctide | 5 μmol/mL | 5 mmol/L | Staurosporine |
| | (1 μmol/mL) | | | |

The readout value of reaction control (complete reaction mixture) was set as a 0% inhibition, and the readout value of background (Enzyme(−)) was set as a 100% inhibition, then the percent inhibition of each test solution was calculated (Table 2).

TABLE 2
Assay Results

| Compound | Kinase | % Inhibition (0.5 μM) | IC₅₀ (M) |
|---|---|---|---|
| A | BTK | 96.9 | 1.25E−08 |
| A | BTK[C481S] | 95.1 | 1.63E−08 |
| B | BTK | 95.6 | 1.25E−08 |
| B | BTK[C481S] | 97.5 | 1.63E−08 |

### Example 2

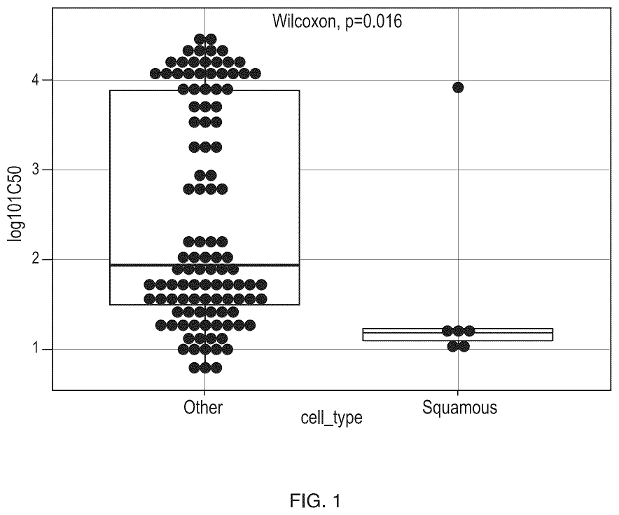

Pan cancer squamous carcinomas displayed high sensitivity to Compound A. For example, FIG. 1 depicts a box and dot plot demonstrating significant sensitivity of pan cancer squamous lines to Compound A (p<0.05). Examples of squamous cell carcinomas contemplated herein may include, but are not limited to, cutaneous squamous cell carcinoma, esophageal squamous cell carcinoma, head and neck squamous cell carcinoma, squamous cell carcinoma of the vulva, and lung squamous cell carcinoma.

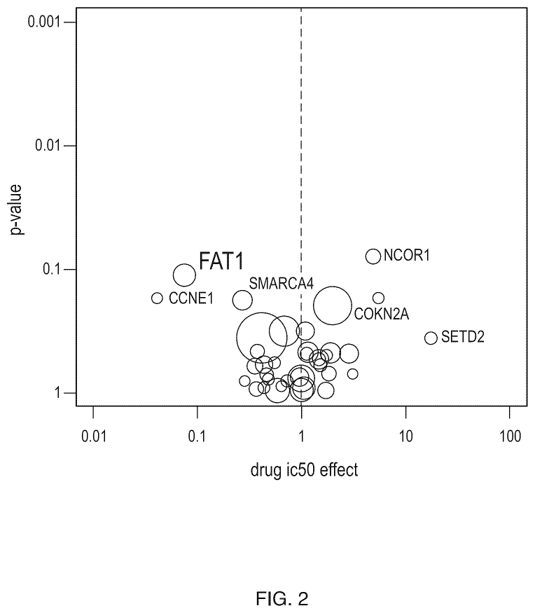

To determine genetic drivers of sensitivity, transformations in 38 cancer genes were observed to be statistically associated with shifts in compound sensitivity across a panel of 102 cell lines, with clear trends emerging. For example, FIG. 2 shows a statistical association of genetic transformations in 38 cancer genes with shifts in sensitivity to Compound a (as measured by ¹⁰logIC₅₀). A shift to the left represents increased sensitivity. Size of circles represents number of cell lines with alteration (minimum of cell lines per alteration).

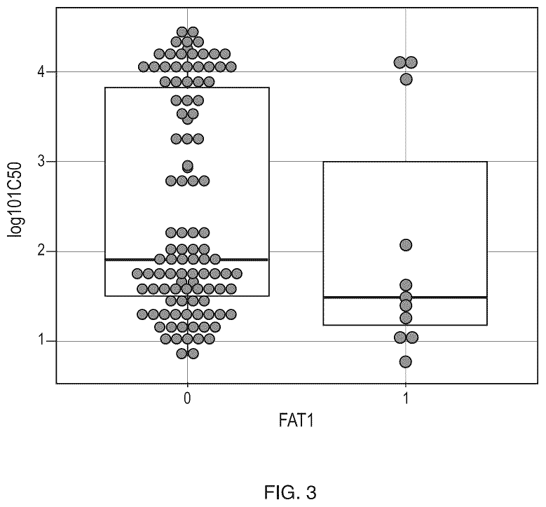

Without wishing to be bound by any particular theory, it is believed that genetic transformations in the FAT1 gene show high prevalence in squamous cell carcinomas. For example, FAT1 is altered (mutations with known significance) in 3% of patients across all cancer types and is highly prevalent in squamous carcinomas, particularly cutaneous and head and neck. FIG. 3 shows a ¹⁰logIC₅₀ distribution of Compound A and FAT1 mutation status. (0=Wild type, 1=Mutant. p=0.16).

### Example 3

The objective of this study was to investigate the antitumor efficacy of orally administered Compound A in the KYSE70 mouse xenograft model for esophageal squamous cell carcinoma.

### Summary

Prior to the in-life phase of the study, the animals were weighed and examined for signs of physical disorder. Animals determined to be suitable were assigned randomly to 1 of 2 treatment groups, Compound A (40 mg/kg) or vehicle control. Administration of Compound A or vehicle orally, once daily, for 28 days. Tumor reads were collected every 2 days up to 28 days, starting treatment at 100 mm³ tumor volume and ending at 2000 mm³ tumor volume. A t-test was applied to determine the significance of differences in tumor volumes.

### Protocol

Compound A was dissolved at 4 mg/mL in vehicle (3 mmol/L sodium citrate buffer, pH 3.0) and administered once daily by oral gavage (100 μL per 10 g mouse weight). Dose materials were prepared weekly in aliquots and stored frozen at −20° C. until dosing. For enrollment of mice into study, subcutaneous tumor volumes were monitored daily until reaching a minimum palpable tumor volume (˜100 mm3 by caliper) in the required number of mice. Tumor-bearing mice were randomized and assigned to each treatment cohort resulting with comparable mean tumor volumes and statistics. Following randomization and study cohort assignment, each dose cohort was treated according to dosage and schedule. Mice were euthanized when study end points were reached.

Tumor volume measurements were performed every 2 days (up to 3 times per week) using a calibrated caliper. Tumor volumes were determined using the formula length (mm)×width (mm)×height (mm)×0.50.

A tumor volume humane endpoint was reached when a 20% loss in body weight (excluding tumor weight) was observed over a week, or a 15% loss in body weight over 2 to 3 day period, or if a body condition score less than 2 (Body Condition Scoring for scoring guide), or the tumor weight exceeded 10% of the non-tumor bearing body weight (tumor weight was calculated based on tumor height×length×width×0.50×density of 1 g/cm³) or when the tumor exceeded 2000 mm³ in size. Body weight measurements were performed every 2 days (up to 3 times per week) during the 28-day treatment period to coincide with tumor volume measurement schedule. A humane endpoint was reached when body weight loss exceeded 20%.

### Results

Treatment with Compound A resulted in substantial tumor growth inhibition. At baseline, average tumor volumes for the vehicle and Compound A groups were 101.4 and 109.8 mm³, respectively; on Day 27, average tumor volumes for the vehicle and Compound A groups were 580.5 and 31.5 mm³, respectively, representing increases in tumor volume of 472% for the vehicle control group and a decrease of 71% for the Compound A group. Student's t-test showed that tumor volumes on Day 27 were significantly lower in the Compound A-treated group versus the vehicle control group (p≤0.001). Mean baseline body weight was 24.3 and 24.5 g in the Compound A and vehicle control groups, respectively. On Day 27, a mean percent increase from baseline in body weight of 2.0% and 3.5% was seen in the Compound A 40 mg/kg and vehicle control group, respectively.

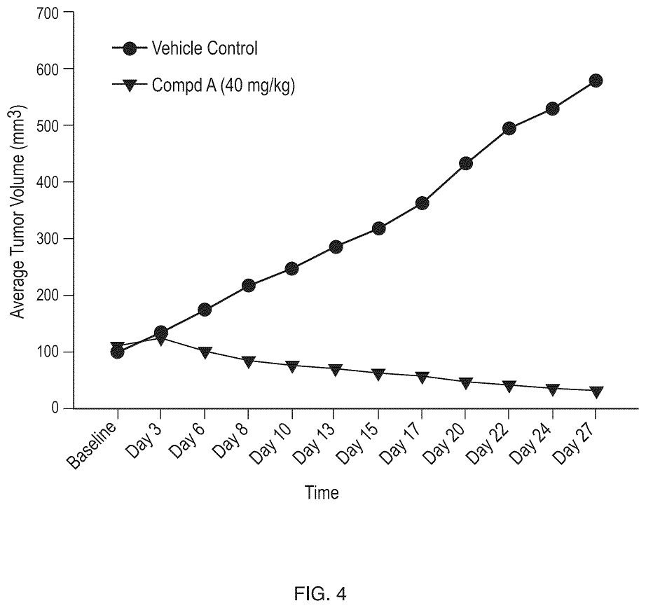

A display of tumor volumes over time is shown in FIG. 4. Table 3 shows relative tumor volume and tumor growth inhibition on Day 27 in the KYSE70 (esophageal squamous cell carcinoma) xenograft model, by treatment group. The abbreviation RTV28 (relative tumor volume, day 28)=[avg. tumor volume D28]/[avg. tumor volume at baseline]. The abbreviation TGI % (percent tumor growth inhibition)=(1−[RTV28 of the treated group]/[RTV28 vehicle])×100.

TABLE 3

| | Avg. Baseline | Avg. Day 28. | Relative Tumor | Tumor Growth |
|---|---|---|---|---|
| | Tumor Volume | Tumor Volume | Volume | Inhibition |
| Treatment Group | (mm³) | (mm³) | (RTV28) | (TGI) |
| Vehicle | 106.1 | 580.5 | 5.77 | — |
| Cmpd A (40 mg/kg) | 106.0 | 31.5 | 0.29 | 95% |

### Example 4

The objective of this study was to investigate the antitumor efficacy of orally administered Compound A in the Cal27 mouse xenograft model for tongue squamous cell carcinoma.

### Summary

Prior to the in-life phase of the study, the animals were weighed and examined for signs of physical disorder. Animals determined to be suitable were assigned randomly to 1 of 2 treatment groups, Compound A (40 mg/kg) or vehicle control. Administration of Compound A or vehicle orally, once daily, for 28 days. Tumor reads were collected every 2 days up to 28 days, starting treatment at 100 mm³ tumor volume and ending at 2000 mm³ tumor volume. A t-test was applied to determine the significance of differences in tumor volumes.

### Protocol

Compound A was dissolved at 4 mg/mL in vehicle (3 mmol/L sodium citrate buffer, pH 3.0) and administered once daily by oral gavage (100 μL per 10 g mouse weight). Dose materials were prepared weekly in aliquots and stored frozen at −20° C. until dosing. For enrollment of mice into study, subcutaneous tumor volumes were monitored daily until reaching a minimum palpable tumor volume (˜100 mm3 by caliper) in the required number of mice. Tumor-bearing mice were randomized and assigned to each treatment cohort resulting with comparable mean tumor volumes and statistics. Following randomization and study cohort assignment, each dose cohort was treated according to dosage and schedule. Mice were euthanized when study end points were reached.

Tumor volume measurements were performed every 2 days (up to 3 times per week) using a calibrated caliper. Tumor volumes were determined using the formula length (mm)×width (mm)×height (mm)×0.50.

A tumor volume humane endpoint was reached when a 20% loss in body weight (excluding tumor weight) was observed over a week, or a 15% loss in body weight over 2 to 3 day period, or if a body condition score less than 2 (Body Condition Scoring for scoring guide), or the tumor weight exceeded 10% of the non-tumor bearing body weight (tumor weight was calculated based on tumor height×length×width×0.50×density of 1 g/cm³) or when the tumor exceeded 2000 mm³ in size. Body weight measurements were performed every 2 days (up to 3 times per week) during the 28-day treatment period to coincide with tumor volume measurement schedule. A humane endpoint was reached when body weight loss exceeded 20%.

### Results

Treatment with Compound A resulted in substantial TGI. At baseline, average tumor volumes for the vehicle and Compound A groups were 116.1 and 116.2 mm3, respectively; on Day 27, average tumor volumes for the vehicle and Compound A groups were 647.2 and 91.4 mm3, respectively, representing an increase in tumor volume of 457% for the vehicle control group and a decrease of 78% for the Compound A group. Student's t-test showed that tumor volumes on Day 27 were significantly lower in the Compound A treated group versus the vehicle control group (p≤0.001). Mean baseline body weight was 23.3 and 23.7 g in the Compound A and vehicle control group, respectively. On Day 27, a mean percent increase from baseline in body weight of 2.1% and 1.8% was seen in the Compound A 40 mg/kg and vehicle control group, respectively.

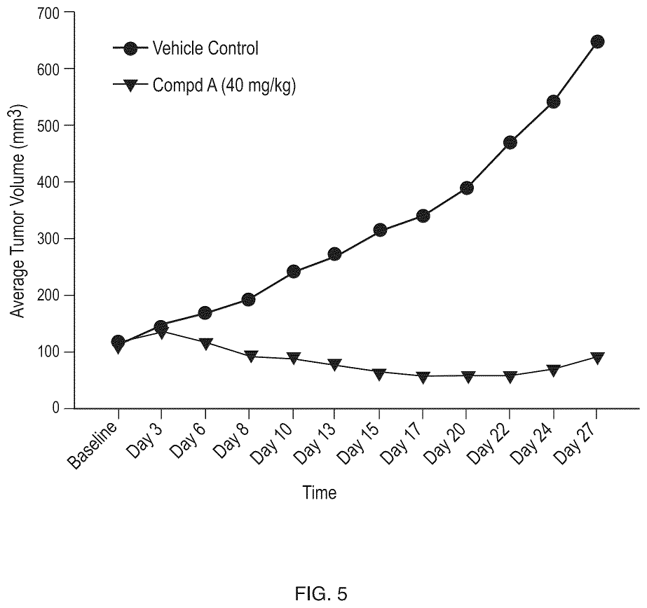

A display of tumor volumes over time is shown in FIG. 5. Table 4 shows relative tumor volume and tumor growth inhibition on Day 27 in the Cal27 (tongue squamous cell carcinoma) xenograft model, by treatment group. The abbreviation RTV28 (relative tumor volume, day 28)=[avg. tumor volume D28]/[avg. tumor volume at baseline]. The abbreviation TGI % (percent tumor growth inhibition)=(1−[RTV28 of the treated group]/[RTV28 vehicle])×100.

TABLE 4

| | Avg. Baseline | Avg. Day 28. | Relative Tumor | Tumor Growth |
|---|---|---|---|---|
| | Tumor Volume | Tumor Volume | Volume | Inhibition |
| Treatment Group | (mm³) | (mm³) | (RTV28) | (TGI) |
| Vehicle | 116.1 | 647.2 | 5.57 | — |
| Cmpd A (40 mg/kg) | 116.2 | 91.4 | 0.78 | 86% |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A method of treating a cancer in a patient in need thereof, comprising administering to the patient an effective amount of a Bruton's tyrosine kinase (BTK) inhibitor represented by Formula I: 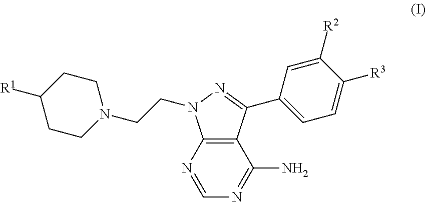
or a pharmaceutically acceptable salt or stereoisomer thereof, wherein:
R¹ is selected from the group consisting of NHR^{a} and NR^{a}R^{b};
R^{a} and R^{b} are each independently selected from the group consisting of C₁-C₆alkyl, C₂-C₆alkenyl, C₃-C₆alkynyl, C₃-C₆cycloalkyl and C₃-C₆cycloalkenyl; or R^{a} and R^{b}, together with the nitrogen to which they are attached, may be joined to form C₃-C₆heterocyclyl;
R² is selected from the group consisting of OR¹¹, hydrogen, halo, NHR¹¹, C₁-C₆alkyl, C₂-C₆alkenyl and C₂-C₆alkynyl;
R³ is selected from the group consisting of NHCO₂R⁴, C₁-C₆alkyl, C₂-C₆alkenyl, C₂-C₆alkynyl, aryl, halo, aryloxy, NH(CO)NR⁵R⁶, NH(CO)R⁷, NH-C₁-C₆alkyl, NH-C₂-C₆alkenyl, NH(CH₂)ₙ-aryl, (CH₂)ₚ-heteroaryl, (CH₂)_{q}CO₂R⁸, (CH₂)ᵣCOR⁹ and NHSO₂R¹⁰; wherein each C₁-C₆alkyl, C₂-C₆alkenyl, aryl or heteroaryl moiety in the aforementioned list is optionally further substituted by one or more groups each independently selected from the group consisting of C₁-C₆alkyl, halo, OH, NR^{c}R^{d}, CONR^{c}R^{d}, C₁-C₆alkoxy, aryloxy, and CO₂H;
R⁴ to R¹¹ are each independently selected from the group consisting of C₁-C₆alkyl, C₂-C₆alkenyl and aryl;
R^{c} and R^{d} are each independently selected from the group consisting of hydrogen, C₁-C₆alkyl and phenyl; and
n, p, q, and r are each independently selected from 0, 1, 2, 3, 4, 5 and 6.

2. The method of claim 1, wherein R^{a} and R^{b} are each independently C₁-C₆alkyl or C₂-C₆alkenyl.

3. The method of claim 1 or 2, wherein R^{a} and R^{b} are each CH₃.

4. The method of claim 1, wherein R^{a} is CH₃ and R^{b} is hydrogen.

5. The method of any one of claims 1-4, wherein R² is C₁-C₆alkoxy or hydrogen.

6. The method of any one of claims 1-5, wherein R² is OCH₃.

7. The method of any one of claims 1-6, wherein R⁴ to R¹¹ are each independently C₁-C₆alkyl.

8. The method of any one of claims 1-7, wherein R³ is selected from the group consisting of NHCO₂-C₁-C₆alkyl, NHCO-C₁-C₆alkyl, NH(CH₂)ₙ-aryl, NHCONH-C₁-C₆alkyl, (CH₂)ₚ-heteroaryl and (CH₂)_{q}CO₂-C₁-C₆alkyl.

9. The method of any one of claims 1-8, wherein R³ is selected from the group consisting of NHCO₂-^{t}Bu, NHCOCH₂C(CH₃)₃, NHCH₂phenyl, NHCONH-^{t}Bu, CH₂-(4-methyl-oxazol-2-yl) and CH₂CO₂-^{t}Bu.

10. The method of any one of claims 1-9, wherein R³ is NHCO₂-^{t}Bu.

11. The method of any one of claims 1-10, wherein the tyrosine kinase inhibitor is selected from the group consisting of 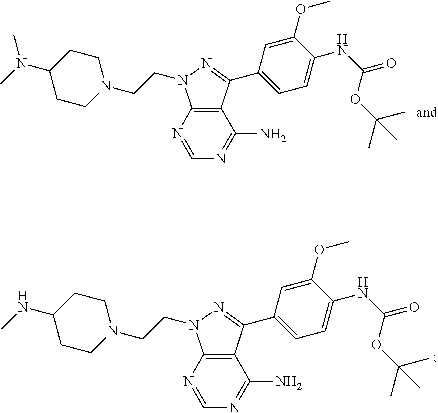
or a pharmaceutically acceptable salt thereof.

12. The method of claim 11, wherein the tyrosine kinase inhibitor is

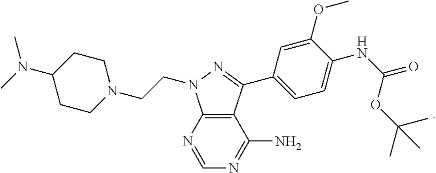 [SMILES: COc1cc(-c2nn(CCN3CCC(N(C)C)CC3)c3ncnc(N)c23)ccc1NC(=O)OC(C)(C)C ; "."]

13. The method of claim 11, wherein the tyrosine kinase inhibitor is

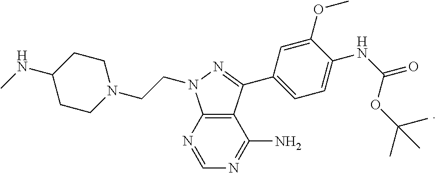 [SMILES: CNC1CCN(CCn2nc(-c3ccc(NC(=O)OC(C)(C)C)c(OC)c3)c3c(N)ncnc32)CC1 ; "."]

14. The method of any one of claims 1-13, wherein the cancer is a B-cell cancer.

15. The method of any one of claims 1-14, wherein the cancer is selected from the group consisting of chronic lymphocytic leukemia, small lymphocytic leukemia, mantle cell lymphoma, non-Hodgkin's lymphoma, marginal zone lymphoma, and Waldenström macroglobulinemia.

16. The method of any one of claims 1-15, wherein the cancer harbors a BTK mutation.

17. The method of claim 16, wherein the BTK mutation is selected from the group consisting of a C481F mutation, a C481G mutation, a C481R mutation, a C481S mutation, and a C481Y mutation.

18. The method of claim 16 or 17 wherein the BTK mutation is a C481S mutation.

19. The method of any one of claims 16-18, wherein the BTK mutation in the cancer is a result of previously treating the cancer with one or more other cancer therapeutic agents.

20. The method of any one of claims 1-19, wherein treating the cancer with the one of more other cancer therapeutic agents is no longer effective in treating the cancer.

21. The method of any one of claims 1-20, wherein the one or more other cancer therapeutic agents is selected from the group consisting of ibrutinib, acalabrutinib, zanubrutinib, fenebrutinib, tirabrutinib, tolebrutinib, evobrutinib, pirtobrutinib, spebrutinib.

22. The method of any one of claims 1-21, wherein the BTK mutation is a heterozygous BTK mutation.

23. The method of any one of claims 1-22, wherein the BTK mutation is a homozygous BTK mutation.

24. The method of any one of claims 1-23, wherein the tyrosine kinase inhibitor is administered orally, subcutaneously, intraperitoneally or intravenously.

25. The method of any one of claims 1-24, wherein the method further and optionally comprises administering one or more additional cancer chemotherapeutic agents.

26. The method of any one of claims 1-25, wherein the method further and optionally comprises administering an additional cancer chemotherapeutic agent.

27. The method of any one of claims 1-26, wherein the tyrosine kinase inhibitor further inhibits SRC family kinases.

28. A method of treating a cancer alleviated by the selective inhibition of BTK in a patient in need thereof, comprising administering to the patient an effective amount of a tyrosine kinase inhibitor represented by: 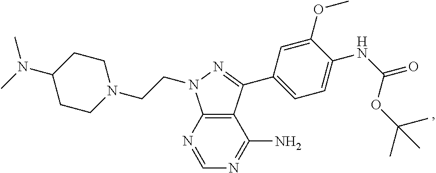 [SMILES: COc1cc(-c2nn(CCN3CCC(N(C)C)CC3)c3ncnc(N)c23)ccc1NC(=O)OC(C)(C)C ; ","]
or a pharmaceutically acceptable salt thereof.

29. The method of claim 28, wherein the cancer is selected from the group consisting of chronic lymphocytic leukemia, small lymphocytic leukemia, mantle cell lymphoma, non-Hodgkin's lymphoma, marginal zone lymphoma, and Waldenström macroglobulinemia.

30. The method of claim 28 or 29, wherein the cancer harbors a BTK mutation.

31. The method of claim 30, wherein the BTK mutation is a C481 mutation.

32. A method of treating a cancer harboring a BTK mutation in a patient in need thereof, comprising administering to the patient an effective amount of a tyrosine kinase inhibitor represented by: 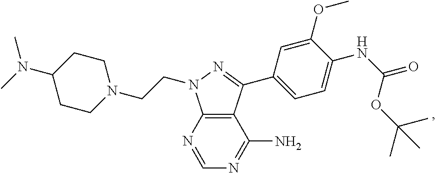 [SMILES: COc1cc(-c2nn(CCN3CCC(N(C)C)CC3)c3ncnc(N)c23)ccc1NC(=O)OC(C)(C)C ; ","]
or a pharmaceutically acceptable salt thereof.

33. The method of claim 32, wherein the cancer is selected from the group consisting of chronic lymphocytic leukemia, small lymphocytic leukemia, mantle cell lymphoma, non-Hodgkin's lymphoma, marginal zone lymphoma, and Waldenström macroglobulinemia.

34. The method of claim 32 or 33, wherein the BTK mutation is a C481 mutation.

35. The method of any one of claims 32-34, wherein the BTK mutation in the cancer is a result of previously treating the cancer with one or more other cancer therapeutic agents.

36. The method of any one of claims 32-35, wherein the one or more other cancer therapeutic agents is selected from the group consisting of ibrutinib, acalabrutinib, zanubrutinib, fenebrutinib, tirabrutinib, tolebrutinib, evobrutinib, pirtobrutinib, spebrutinib.

37. A method of treating a B-cell cancer harboring a BTK C481 mutation in a patient in need thereof, comprising administering to the patient an effective amount of tert-butyl (4-(4-amino-1-(2-(4-(dimethylamino)piperidin-1-yl)ethyl)-1H-pyrazolo[3,4-d]pyrimidin-3-yl)-2-methoxyphenyl)carbamate, or a pharmaceutically acceptable salt thereof, wherein the cancer is resistant to treatment with ibrutinib, acalabrutinib, zanubrutinib, fenebrutinib, tirabrutinib, tolebrutinib, evobrutinib, pirtobrutinib, and/or spebrutinib.

38. A method of treating squamous cell carcinoma in a patient identified as having said carcinoma, and in need of treatment, comprising administering to the patient an effective amount of a tyrosine kinase inhibitor represented by Formula I: 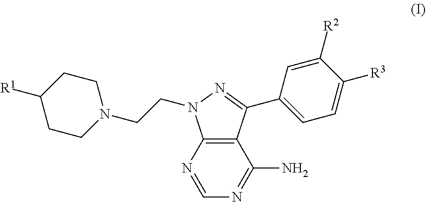
or a pharmaceutically acceptable salt or stereoisomer thereof, wherein:
R¹ is selected from the group consisting of NHR^{a} and NR^{a}R^{b};
R^{a} and R^{b} are each independently selected from the group consisting of C₁-C₆alkyl, C₂-C₆alkenyl, C₃-C₆alkynyl, C₃-C₆cycloalkyl and C₃-C₆cycloalkenyl; or R^{a} and R^{b}, together with the nitrogen to which they are attached, may be joined to form C₃-C₆heterocyclyl;
R² is selected from the group consisting of OR¹¹, hydrogen, halo, NHR¹¹, C₁-C₆alkyl, C₂-C₆alkenyl and C₂-C₆alkynyl;
R³ is selected from the group consisting of NHCO₂R⁴, C₁-C₆alkyl, C₂-C₆alkenyl, C₂-C₆alkynyl, aryl, halo, aryloxy, NH(CO)NR⁵R⁶, NH(CO)R⁷, NH-C₁-C₆alkyl, NH-C₂-C₆alkenyl, NH(CH₂)ₙ-aryl, (CH₂)ₚ-heteroaryl, (CH₂)_{q}CO₂R⁸, (CH₂)ᵣCOR⁹ and NHSO₂R¹⁰; wherein each C₁-C₆alkyl, C₂-C₆alkenyl, aryl or heteroaryl moiety in the aforementioned list is optionally further substituted by one or more groups each independently selected from the group consisting of C₁-C₆alkyl, halo, OH, NR^{c}R^{d}, CONR^{c}R^{d}, C₁-C₆alkoxy, aryloxy, and CO₂H;
R⁴ to R¹¹ are each independently selected from the group consisting of C₁-C₆alkyl, C₂-C₆alkenyl and aryl;
R^{c} and R^{d} are each independently selected from the group consisting of hydrogen, C₁-C₆alkyl and phenyl; and
n, p, q, and r are each independently selected from 0, 1, 2, 3, 4, 5 and 6.

39. The method of claim 38, wherein R^{a} and R^{b} are each independently C₁-C₆alkyl or C₂-C₆alkenyl.

40. The method of claim 38 or 39, wherein R^{a} and R^{b} are each CH₃.

41. The method of claim 38, wherein R^{a} is CH₃ and R^{b} is hydrogen.

42. The method of any one of claims 38-41, wherein R² is C₁-C₆alkoxy or hydrogen.

43. The method of any one of claims 38-42, wherein R² is OCH₃.

44. The method of any one of claims 38-43, wherein R⁴ to R¹¹ are each independently C₁-C₆alkyl.

45. The method of any one of claims 38-44, wherein R³ is selected from the group consisting of NHCO₂-C₁-C₆alkyl, NHCO-C₁-C₆alkyl, NH(CH₂)ₙ-aryl, NHCONH-C₁-C₆alkyl, (CH₂)ₚ-heteroaryl and (CH₂)_{q}CO₂-C₁-C₆alkyl.

46. The method of any one of claims 38-45, wherein R³ is selected from the group consisting of NHCO₂-^{t}Bu, NHCOCH₂C(CH₃)₃, NHCH₂phenyl, NHCONH-^{t}Bu, CH₂-(4-methyl-oxazol-2-yl) and CH₂CO₂-^{t}Bu.

47. The method of any one of claims 38-46, wherein R³ is NHCO₂-^{t}Bu.

48. The method of any one of claims 38-47, wherein the tyrosine kinase inhibitor is selected from the group consisting of: 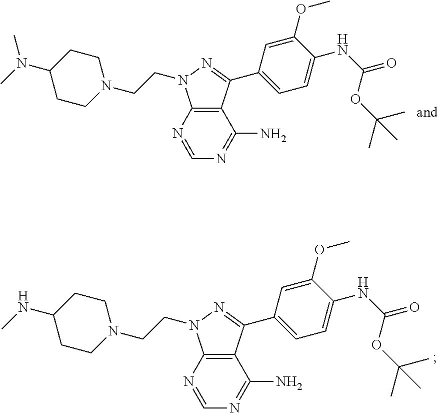
or a pharmaceutically acceptable salt thereof.

49. The method of any one of claims 38-48, wherein the tyrosine kinase inhibitor is

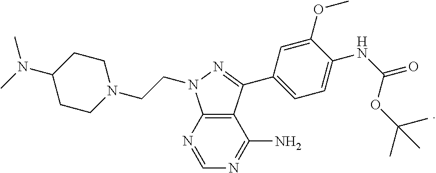 [SMILES: COc1cc(-c2nn(CCN3CCC(N(C)C)CC3)c3ncnc(N)c23)ccc1NC(=O)OC(C)(C)C ; "."]

50. The method of any one of claims 38-49, wherein the squamous cell carcinoma is selected from the group consisting of cutaneous squamous cell carcinoma, esophageal squamous cell carcinoma, tongue squamous cell carcinoma, head and neck squamous cell carcinoma, squamous cell carcinoma of the vulva, and lung squamous cell carcinoma.

51. A method of treating squamous cell carcinoma in a patient identified as having said carcinoma, and in need of treatment, comprising administering to the patient an effective amount of a tyrosine kinase inhibitor represented by 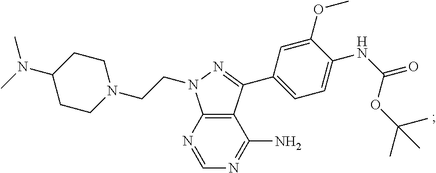 [SMILES: COc1cc(-c2nn(CCN3CCC(N(C)C)CC3)c3ncnc(N)c23)ccc1NC(=O)OC(C)(C)C ; ";"]
or a pharmaceutically acceptable salt thereof;
wherein the squamous cell carcinoma is selected from the group consisting of cutaneous squamous cell carcinoma, esophageal squamous cell carcinoma, tongue squamous cell carcinoma, head and neck squamous cell carcinoma, squamous cell carcinoma of the vulva, and lung squamous cell carcinoma.
